(19) United States
(12) Patent Application Publication
(10) Pub. No.: US 2014/0261431 A1
(43) Pub. Date: Sep. 18, 2014

(54) POSITIVE-PRESSURE RESPIRATORY MASK

(71) Applicant: Opportunity Innovations, LLC, Henrico, VA (US)

(72) Inventor: W. Paul Murphy, Henrico, VA (US)

(73) Assignee: Opportunity Innovations, LLC, Henrico, VA (US)

(21) Appl. No.: 14/203,887

(22) Filed: Mar. 11, 2014

Related U.S. Application Data
(60) Provisional application No. 61/782,118, filed on Mar. 14, 2013.

(51) Int. Cl. A61M 16/06; A61B 17/34; A61M 16/00

(52) U.S. Cl. CPC A61M 16/0622; A61M 16/0057; A61B 17/3423
USPC 128/205.25; 128/206.21; 128/206.28; 128/206.26; 128/206.24

(57) ABSTRACT

A positive-pressure respiratory mask can be used during medical procedures to provide oxygen to a patient while the instruments and devices used in the procedure remain in place. A flap system that includes a flexible diaphragm is used on the mask to create an airtight seal around the instrument.

### CROSS-REFERENCE TO RELATED APPLICATION

This application claims the benefit of U.S. Provisional Application No. 61/782,118, filed Mar. 14, 2013, the contents of which are incorporated herein by reference.

### FIELD OF THE INVENTION

The present invention relates generally to positive-pressure respiratory masks, and more particularly to respiratory masks that can be used with protruding nasal and oral devices in place, such as during surgery or a medical procedure.

### BACKGROUND OF THE INVENTION

A typical positive-pressure respiratory mask is comprised of a hard inner shell, a connecting port for a breathing system, and a flexible outer rim that creates an airtight seal around the nose and mouth. This seal allows the respiratory provider to deliver a pressurized respiratory gas (most commonly oxygen and anesthetic vapors) in a controlled fashion. This is done for two main reasons: first, preparation for starting anesthesia; and, second, providing assisting (or “rescue”) breaths to patients who are unable to breathe effectively for themselves.

The seal between the mask and the face must be tight enough to allow positive pressure gases introduced into the mask to be directly transmitted to the patient's mouth and/or nose without leakage of the gas between the mask and face. When devices are already extending out of the nose or mouth, such as devices being used in the medical procedure, those devices prevent an adequate seal and thus prevent the proper delivery of pressurized gases.

Anesthetic medications provide pain relief, muscle relaxation, amnesia (loss of memory), and reduction of the body's natural responses to pain. These medications also tend to reduce or eliminate the patient's natural respiratory ability. An effective mask seal allows the patient's lungs to be filled with 100% oxygen (rather than the 21% oxygen in the earth's atmosphere) to give a margin of safety prior to giving the anesthetic medications. A typical patient has only enough oxygen in the air in his lungs to support life for a minute or two without breathing, but preparing a patient with extra oxygen has been shown to extend this period of safety to as long as ten minutes. This potentially lifesaving extra oxygen can only be delivered effectively when a tight seal exists around the nose and mouth.

Assistance with breathing is only possible with an airtight seal. The mask seal allows the delivery of artificial breaths with positive pressure from various known devices such as mechanical or manual bellows or pumps into the mask. Since the patient is often unconscious or otherwise unable to breathe effectively, the airtight seal allows the air to be forced through the mouth and/or nose and into the lungs. Without this tight seal, the pressurized air leaks out of the mask and cannot be delivered to the lungs.

In summary, these two common situations require a tight seal around the nose and mouth to deliver the desired gas without room air entering the mixture or the pressurized gas escaping into the environment rather than entering the lungs.

Many patients need breathing assistance while devices are already extending out of the nose or mouth that prevent an adequate seal. These devices may include tubes for feeding, hydration or constant drainage of stomach contents, or equipment like endoscopes or ultrasound probes used during diagnostic or therapeutic procedures on the nose, throat, sinus passages, windpipe, esophagus, stomach, duodenum, or pancreas. Since the known masks cannot provide an adequate seal around these additional devices, providers are unable to deliver breathing assistance with the tubes in place.

The current solutions to these problems are less than ideal. One solution requires the urgent removal of the tube or device from the nose or mouth, terminating the therapy or procedure while breathing assistance is delivered. The second solution is to simply withhold necessary breathing assistance from a patient in urgent need while the ostensibly helpful procedure is continued. Both of these options place already endangered patients at significant additional risk.

A number of prior attempts have been made to address this issue. None of the solutions has proven satisfactory. They either do not permit the addition of the mask over instruments and devices that are already in place within the patient's mouth or nose, or they do not permit a sufficient seal to be maintained to provide positive air pressure to the patient.

Therefore, there is a need for a device that allows the respiratory provider to create a seal around the protruding tubes or equipment without disturbing or removing the protruding device(s).

### SUMMARY OF THE INVENTION

The invention relates to various exemplary embodiments, including devices, products, and methods of treatment, and of using the same.

These and other features and advantages of exemplary embodiments of the invention are described below with reference to the accompanying drawings.

### BRIEF DESCRIPTION OF THE DRAWINGS

FIG. 1 is a perspective view of a respiratory mask according to the present invention.

FIG. 2 is a view of the underside of a mask as in FIG. 1.

FIG. 3 a perspective view of the mask of FIG. 1 with an instrument protruding therefrom.

FIGS. 4A-4C show the mask of FIG. 1 with a flap and diaphragm.

FIGS. 4D-4G show various configurations of the diaphragm of the mask of FIG. 1.

FIGS. 5A-5B show the diaphragm of the mask of FIG. 1 providing a seal around an instrument.

FIGS. 5C-5E show overlapping areas of the diaphragm of the mask of FIG. 1.

### DETAILED DESCRIPTION

In the following detailed description, numeric values and ranges are provided for various aspects of the implementations described. These values and ranges are to be treated as examples only and are not intended to limit the scope of the claims. In addition, a number of materials and ingredients are identified as suitable for various facets of the implementations. These materials and ingredients are to be treated as exemplary and are not intended to limit the scope of the claims.

The invention relates to a respiratory mask that can be kept in place and provide oxygen to a patient who is undergoing a medical procedure that requires medical instruments to be inserted into the nose, the mouth, or both. The mask permits a seal to be maintained over the patient's nose and mouth to provide positive pressure to assist with breathing, while at the same time keeping the instruments in place so that the procedure is not interrupted.

The device incorporates various ways of allowing a device to pass through the outer air-filled sealing rim of the mask and become positioned securely within an airtight seal in the center of the mask's rigid central section.

Referring to FIG. 1, the respiratory mask 100 according to the present invention includes a ‘C-shaped’ air-filled chamber 110 encircling the edge of the mask's rigid shell 120. The chamber 110 is not an unbroken ring; rather the chamber 110 extends fully around the edge of the rigid shell 120, but allows a flexible passageway 130 for indwelling tubes/devices to be passed through the chamber 110 circumference into the rigid central shell portion 120 of the mask 100, and then sealing around the device by the diaphragm 190 in the center.

The C-shaped air-filled chamber 110 produces an airtight seal between the edge of the mask 100 and the patient's face. Specifically, this seal is created using an overlapping flap system 150 that is held in place by the pressure placed on the mask by the provider and the pressurized gas flowing into the mask. Mask 100 can also include plastic prongs 115. The prongs 115 (discussed in more detail below) can be used to strap the mask onto the patient's head to hold it tightly against the patient's face to create the airtight seal. Air can be added to the outer ring of the mask 100 via entry 170 or a similar inlet to adjust the air in the ring.

Referring to FIG. 2, the overlapping flap system 150 can include flexible flaps 152, 154 that form a door to seal around the opening and any instruments contained therein. In one implementation, the flexible flaps 152, 154 may include opposing ‘fish-scales’ that include internal flexible ridges placed in overlapping layers within both the rigid central shell as well as the flexible air-filled tubing around the perimeter of the mask. These flaps 152, 154 allow the provision of an airtight seal despite a gap 130 in the rigid mask body while maintaining the rigidity of the mask body 120 necessary for adequate assistance with ventilation. This overlap thus allows devices of various sizes and locations (such as the patient's nose, mouth, etc.) to be accommodated through the mask's periphery while allowing an airtight seal between the mask 100 and the patient's face once the device has been passed into the central region of the mask. The devices are sealed in place with the diaphragm 190. The mask body 120 includes a gas inlet port or circuit adapter 160 where a source of air or oxygen would be administered to the patient through the mask 100.

FIG. 3 shows an implementation of the mask 100 with an instrument 200 sealed in place by flap system 150 to create a seal around the instrument 200. Air can be added to the outer ring of the mask 100 via entry 170 or a similar inlet to adjust the air in the ring. The flap system 150 can be sealed in place or held by a health care provider present during the procedure to provide positive pressure to permit air into the lungs of the patient while instrument 200 remains in place. Flaps 152, 154 overlap to provide the airtight seal while the instrument 200 is in place. The seal provided by the flaps 152, 154 permits air or oxygen would be administered to the patient through a gas inlet port or circuit adapter 160. To remove the instrument 200, the flaps 152, 154 can be moved or opened so that the instrument 200 can be withdrawn.

Referring to FIG. 4, various ways to form a seal around the protruding device(s) once passed through the circumferential seal into the rigid central portion 120 of the mask 100 are shown. FIG. 4A shows mask 100 with flap system 150 and an area of internal overlap for diaphragm 400 to help create the seal. FIG. 4B shows flap system 150 with diaphragm 400 in an open position so that an instrument or other device may be inserted. In this case, flap 152 is folded down and pivots internally, permitting the slit to twist and open the flap system 150. The flap system 150 can also include an optional latch 155 for maintaining the flap 152 in a closed position. The latch 155 would typically be formed of plastic and may include a post and a rectangular block that rotates about the post. The block could be pivoted about the post by the medical professional to close or open the flap 152 as desired. Other types of latches could also be used as may be within the ordinary skill of those in the art.

FIG. 4C shows mask 100 with flap 152 closed around device 200 protruding from the mask 100. In this case, flap system 150 creates an airtight seal around device 200.

FIGS. 4D-4G show diaphragm 420 in various configurations. In FIG. 4D, overlapping layers of diaphragm 420 are shown. In FIG. 4E, the flap system has opened and diaphragm 420 has opened with it to permit a device to enter the mask. FIG. 4F shows device 200 inserted within diaphragm 420, which closes tightly around device 200 to create an airtight seal. FIG. 4G shows a cross-section of the diaphragm 420 when device 200 is secured within. The mobile portion of diaphragm 422 can be opened, but is shown closed, and it overlaps the non-mobile portion of diaphragm 424 to create the airtight seal around device 200.

The diaphragms used herein can be formed of various flexible plastic diaphragms that allow an airtight seal with or without a device inserted through them, as well as other rigid pivoting disks integrally connected to the rigid portion of the mask that would allow a more customized seal around the protruding device(s).

FIG. 5 shows a cutaway view of a diaphragm 500 for use with the mask of the present invention. As shown in FIGS. 5A-5B, the diaphragm 500 provides a seal around an instrument 510 that is used during medical or surgical procedures. In this way, the mask is still able to be used to provide rescue breaths to the patient while the procedures are taking place. The diaphragm 500 provides an airtight seal around instruments 510 having smaller or larger diameters, as needed. FIGS. 5C-5D show the diaphragm 500 having an overlapping area. As shown in FIG. 5E, this overlapping area 540 can be used to form the airtight seal around the instrument 510 that is shown in FIG. 5B.

In typical use, if a patient is in need of breathing assistance or “rescue breaths” during a procedure where tools or instruments are protruding from the patient's nose or mouth, the medical professional would open the flap system of the mask and place it over the instrument to create the airtight seal. The mask would be sealed by the flexible diaphragm. The mask may be held tightly against the patient's face by the hand(s) of the medical professional, or it may be secured using mask straps that would attach to 4 small plastic prongs spaced equally around, and integrated into, the central accommodation port. These prongs would be incorporated into the rigid central frame of this mask, as previously described mask art use a circular ring that encompasses the central area of the mask, which would prevent the protruding device from passing through the side of the mask into the central accommodation port. Oxygen or air could be inserted into the mask; the positive pressure would force air into the patient's lungs.

While the invention has been described in conjunction with specific exemplary implementations, it is evident to those skilled in the art that many alternatives, modifications, and variations will be apparent in light of the foregoing description. Accordingly, the invention is intended to embrace all such alternatives, modifications, and variations that fall within the scope and spirit of the appended claims.

What is claimed is:

1. A respiratory mask comprising:
a mask shell having a slit or opening that forms a flexible passageway; and
a flap system within the flexible passageway that forms an airtight seal over the passageway.

2. The respiratory mask of claim 1, wherein the mask shell forms a C-shaped airtight air-filled chamber when placed over a patient's nose and mouth.

3. The respiratory mask of claim 1, wherein the flap system comprises flexible flaps that form a door.

4. The respiratory mask of claim 3, wherein the flexible door is formed of overlapping layers to provide the airtight seal over the passageway.

5. The respiratory mask of claim 4, further comprising flexible air-filled tubing around a perimeter of the mask shell.

6. The respiratory mask of claim 5, wherein the flexible door includes internal flexible ridges that form the airtight seal with the flaps and the air-filled tubing.

7. The respiratory mask of claim 3, wherein the flexible flaps are configured to form an airtight seal over a medical instrument protruding from the flexible passageway.

8. A respiratory mask comprising:
a mask shell configured to fit over a patient's nose and mouth, the shell having a slit or opening that forms a flexible passageway, the passageway configured to permit a medical instrument to protrude therefrom; and
a diaphragm covering the flexible passageway and forming an airtight seal over the passageway and any medical instrument that protrudes therefrom.

9. The respiratory mask of claim 8, wherein the diaphragm includes overlapping layers.

10. The respiratory mask of claim 8, wherein the diaphragm is formed of flexible plastic.

11. The respiratory mask of claim 8, wherein the diaphragm is configured to open to fit around the medical instrument.

12. A method of using the respiratory mask of claim 8, which comprises:
opening the diaphragm to fit around the medical instrument;
placing the mask shell over the patient's nose and mouth; and
closing the diaphragm around the medical instrument to form an airtight seal over the patient's nose and mouth and around the medical instrument.

13. The method of claim 12, further comprising holding the mask in place on the patient's face to maintain the airtight seal.

14. The method of claim 12, further comprising securing the mask in place on the patient's face by straps that fit around the patient's head.

15. The method of claim 12, further comprising attaching a supply of air or oxygen to the mask to provide breathing assistance to the patient.

16. A respiratory mask comprising:
a mask shell configured to fit over a patient's nose and mouth, the shell having a slit or opening that forms a flexible passageway, the passageway configured to permit a medical instrument to protrude therefrom; and
a flexible flap system within the flexible passageway; and
a diaphragm covering the flexible passageway and forming an airtight seal over the passageway and any medical instrument that protrudes therefrom.

17. The respiratory mask of claim 16, wherein the flap system comprises one or more flaps and a latch for maintaining the flaps in an open or closed configuration.
